(19) United States
(12) Patent Application Publication
(10) Pub. No.: US 2022/0267423 A1
(43) Pub. Date: Aug. 25, 2022

(54) IGE ANTIBODIES CROSS-REACTIVE TO ALLERGENS AND TUMOR ANTIGENS

(71) Applicant: IgGenix, Inc., South San Francisco, CA (US)

(72) Inventor: Richard Boismenu, South San Francisco, CA (US)

(21) Appl. No.: 17/391,314

(22) Filed: Aug. 2, 2021

Related U.S. Application Data
(60) Provisional application No. 63/152,181, filed on Feb. 22, 2021.

(51) Int. Cl. C07K 16/18; C12N 15/86

(52) U.S. Cl. CPC C07K 16/18; C12N 15/86; C07K 2317/33; C12N 2750/14143

(57) ABSTRACT

The invention relates to cross-reactive antibodies or antigen-binding portions thereof with specificity for at least one non-tumor antigen and a tumor antigen.

### TECHNICAL FIELD

The invention generally relates to the fields of medicine and cancer, and, more particularly, to therapeutic methods for treating cancer.

### BACKGROUND

Cancers are attributed to nearly 10 million deaths globally. In the early stages of cancer, immune cells may be capable of eliminating individual cancer cells as they arise. However, as time goes on, cancer cells can develop genetic changes that allow them to evade the immune system.

As is generally understood, an allergy is an immune response to a type of antigen that produces an abnormally vigorous response in which the immune system fights off a perceived threat that would otherwise be harmless. In one aspect, an allergen is an antigen that is capable of stimulating a type-I hypersensitivity reaction in atopic individuals through Immunoglobulin E (IgE) responses. Most humans mount significant IgE responses only as a defense against parasitic infections. However, some individuals may respond to many common environmental antigens. This hereditary predisposition is called atopy. In atopic individuals, non-parasitic antigens stimulate inappropriate IgE production, leading to type I hypersensitivity.

Immunoglobulin E (IgE) antibodies mediate the allergic response. They bind to specific receptors on inflammatory immune cells, including mast cells in mucosal tissues lining body surfaces and cavities, as well as basophils in the circulation. These cells mediate allergic responses triggered by specific antigens (allergens) that are recognized by IgE through the release of inflammatory molecules, such as histamine. The inflammatory response is responsible for symptoms, such as sneezing, runny or stuffed nose, itchy eyes, breathing difficulties, and, in extreme cases, anaphylactic shock and even death.

New epidemiologic studies support an inverse association between allergy or atopy and cancer risk. However, the biological mechanisms connecting atopy and cancer remain elusive. As a result, no therapeutics methods for treating cancer based on atopy have advanced.

As a result, drug therapies, such as checkpoint inhibitors, have been needed to generate an immune response against cancer cells. Due to the complexity and heterogeneity of cancer cells, however, there is no guarantee that any particular drug therapy will successfully result in remission and control of a patient's cancer. Moreover, remission and control can be fleeting, with drug targets changing as cancer cells continue to mutate and develop resistances to previously effective therapies.

### SUMMARY

The present invention provides therapeutic methods for treating cancer using modified cross-reactive antibodies. Compositions and methods of the invention utilize IgE antibodies from atopic/allergic individuals that have anti-tumor activity. In one embodiment, IgE sequences identified from B cells obtained from an allergic or atopic individual are expressed as recombinant IgE molecules or isotype-switched IgG molecules. In an alternative embodiment, functional antibodies are expressed as recombinant proteins and screened against cancer cell lines for specific binding to cancer cells types. The invention can be carried out with whole antibodies or fragments thereof. Moreover, as discussed herein, the invention contemplates Fc variants, non-Ig components, drug conjugates (e.g., antibody-drug conjugates) and other binding derivatives that have the effect of mounting an immune-like response against a tumor.

Antibodies or antibody-binding portions derived from antibodies according to the invention may be monoclonal or polyclonal antibodies. Moreover, the invention contemplates a mixture of monoclonal antibodies or antibodies derived from polyclonal serum. The antibodies may include a heavy chain variable region sequence, and a light chain variable region sequence derived from an IgE-producing human B cell associated with at least one non-tumor antigen.

Antibodies (or antigen-binding portions thereof) according to the invention are able to generate an “immune response” against cancer cells. Antibodies for use in the invention are derived from IgE antibodies produced in response to an allergic reaction. For example, useful antibodies are derived from a food allergen, a plant allergen, a fungal allergen, an animal allergen, a drug allergen, a cosmetic allergen, and a latex allergen. The food allergen may be selected from the group consisting of a milk allergen, an egg allergen, a nut allergen, a fish allergen, a shellfish allergen, a soy allergen, a legume allergen, a seed allergen, and a wheat allergen.

As used herein, “antibodies or antibody” is assumed to include a whole antibody or an antigen-binding fragment thereof. Antibodies of the invention may be delivered in any suitable therapeutic form. For example, antibodies may be delivered in any acceptable carrier, such as a nanoparticle, including a lipid nanoparticle, or in conjugation with a carrier molecule. In addition, antibodies may be delivered as nucleic acid (either RNA or DNA) encoding the antibody or a portion thereof. Nucleic acid can be delivered via any suitable vector, including a viral vector, such as an adeno-associated virus (AAV). Nucleic acid encoding antibodies of the invention can also be delivered via CRISPR-Cas induced genomic modification. Formulations contain pharmaceutically-acceptable diluents, adjuvants and carriers and may include stability enhancing elements to increase bio-availability and serum half-life. Moreover, formulations may contain elements that assist in tumor eradication. For example, antibodies of the invention may be delivered as antibody-drug conjugates comprising an antibody of the invention linked to a cytotoxic element. In this way, the formulation is designed to induce an immune response to the tumor, while also introducing a cytotoxin to the tumor environment. Formulations of the invention are also useful as an adjunct to other chemotherapeutic approaches.

Antibodies of the invention are also useful for diagnostic purposes. In one example, antibodies of the invention are used in tumor imaging. In a preferred embodiment, an IgE sequence isolated from B cells obtained from an allergic/atopic individual are expressed as recombinant IgE (or isotype-switched IgG) molecules to which a detectable label is attached. The detectable label can be any appropriate label, such as a radiolabel, a fluorescent label or other suitable image marker. The recombinant labeled antibody is then delivered to a tumor to which it binds and appropriate imaging is done. The imaging may be accomplished in vivo, in vitro or ex vivo.

Antibodies of the invention are useful to monitor prognosis and recurrence, to stage disease and to drive therapeutic selection. For example, levels of antibody engagement are important in determining the stage of disease and/or disease progression or recurrence. The type(s) of antibodies that bind a tumor are useful not only in assessing disease severity and progression but also in terms of therapeutic selection.

Aspects of the invention include methods of preventing or treating cancer with formulations comprising antibodies of the invention. Certain methods comprise administering a therapeutically effective amount of a pharmaceutical formulation comprising a naked antibody or fragment. In addition, the antibody or fragment may be delivered via a vector, a lipid nanoparticle, or other carrier. Antibody may also be delivered as nucleic acid encoding the antibodies or antigen-binding fragments.

For example, the invention may include transducing one or more cells with the nucleic acid encoding a cross-reactive antibody or antigen-binding portion thereof, thereby creating one or more transduced host cells capable of expressing the cross-reactive antibodies or antigen binding portion thereof. Methods further comprise expressing the antibodies or antigen binding portions. Any suitable host cell will work, including engineered host cells for delivery of constructs of the invention. The nucleic acid may be DNA or RNA or modifications thereof. Finally, a CRISPR/Cas system with appropriate guide RNAs may be used to integrate sequence expressing engineered cross-reactive antibodies of the invention.

Aspects of the invention include screening assay for the binding of cross-reactive antibodies derived from atopic individuals to diverse panels of commercially available allergens and assaying the antibodies cross-reactivity for cancer. Allergens may be expressed for assays as recombinant proteins using well-established molecular biology procedures. Recombinant antibodies may be derived from atopic individuals' IgE sequences. Tumor cells for assays may be frozen and/or fixed tumor slices, and tumor organoids.

Whenever the term, “antibody” is used in the disclosure it is intended to mean polyclonal antibodies, monoclonal antibodies, or antigen-binding portions or fragments of any of the foregoing. In one embodiment, the antibodies are isotype-switched IgG or IgM antibodies or antigen-binding fragments thereof having a binding specificity to an associated non-tumor antigen obtained from an IgE antibody while being cross-reactive to cancer to thereby afford protection (i.e., prevent or suppress cancer) by promoting an immune response against the cancer cells.

The production of high-affinity, allergen-specific antibodies or fragments may include in vivo production via a vector, such as a viral vector, Cas-mediated introduction in host cells, including modified bacterial cells or epithelial cells in, for example, the gut, or by other means for the production/expression of the cross-reactive antibodies. Antibodies may be expressed from host cells into which nucleic acids encoding the cross-reactive antibody or antigen-binding fragment thereof are introduced. The expressed cross-reactive antibody may include at least one heavy chain variable region sequence or light chain variable region sequence derived, for example, from an IgE-producing human B cell and/or an IgG producing human B. Compositions of the invention may be delivered as protein or as nucleic acid and may be delivered by any suitable means. Moreover, compositions of the invention may be combined with acceptable diluents, carriers, and adjuvants. Thus, in a preferred embodiment, antibodies for use in the invention are class-switching antibodies in which a portion of an IgE antibody is swapped into an IgG antibody as described herein.

An antibody, or antigen-binding fragment thereof, for use in the invention is capable of binding to a known non-tumor allergen. For example, the specific allergen may include, but is not limited to, a food allergen, a plant allergen, a fungal allergen, an animal allergen, a dust mite allergen, a drug allergen, a cosmetic allergen, or a latex allergen. In some embodiments, antibodies specifically bind to a food allergen, such as a milk allergen, an egg allergen, a nut allergen, a fish allergen, a shellfish allergen, a soy allergen, a legume allergen, a seed allergen, or a wheat allergen. In some embodiments, antibodies specifically bind to a peanut allergen. Advantageously, these same antibody or antigen-binding fragments are cross-reactive to cancer cells.

In some embodiments, antibodies of the invention are delivered directly in a prolonged release formulation. The antibody itself may be modified to include features that increase serum half-life. Antibodies may be pegylated, conjugated to other proteins (e.g., human serum albumin) or provided in a vehicle that causes delayed release of the antibody.

Therapeutic compositions of the invention may comprise an antibody, or antigen-binding portion thereof, formulated for delivery. Delivery may be in oral, intravenous, aerosol or other appropriate formulations. Alternatively, therapeutic compositions of the invention may be delivered in the form of a nucleic acid encoding an appropriate antibody or antigen-binding portion thereof.

### DETAILED DESCRIPTION

The present invention is directed to methods for treating, diagnosing, staging and monitoring cancer.

The present invention relates to novel anti-cancer antibodies derived from IgE antibodies obtained from allergic/atopic individuals. In a preferred embodiment, IgE antibodies (or nucleic acid encoding them) are identified from an allergic/atopic individual's IgE-positive B cells. Those antibodies are then screened for their ability to bind to one or more antigen(s) presenting on a selected tumor or class of tumor. In a highly-preferred embodiment, nucleic acid from atopic/allergic IgE-positive B cells is expressed as recombinant IgE, or isotype switched IgG or IgM molecules. Those recombinant molecules are screened against panels of known or previously unknown tumor antigens for their binding ability. Recombinant molecules can be screened against any appropriate cancer cell line, primary human tumor, frozen or formalin-fixed tumor samples, tumor organoids or isolated single cells. Any recombinant molecules so identified are formulated for use as cancer therapeutics, either alone or as an adjunct to other cancer therapies, including but not limited to immunotherapies.

In one embodiment, the identified therapeutic is an immunoglobulin Fc variant or other immunoglobulin fragment that is capable of binding to a tumor cell surface molecule. Antigen-binding derivatives of molecules discovered via the screening methods outlined herein are also useful directly as therapeutics or as adjuncts to conventional chemotherapies. In some embodiments, identified recombinant molecules are administered as naked antibodies that bind to a tumor cell in order to reduce the ability of the tumor cell to grow (e.g., by blocking growth factors). Alternatively, binding may attract monophages or natural killer cells which interact with the Fc portion of the immunoglobulin molecule to destroy the tumor cell.

In an alternative embodiment, immunoglobulin molecules obtained from IgE-positive B cells are screened directly against panels of cancer cell lines for binding. Immunoglobulins that bind are then formulated as potential therapeutics. In some embodiments, the identified IgE immunoglobulins are modified or fragmented for optimal use. For example, solely an antigen-binding fragment may be produced for use as a therapeutic. Alternatively, useful immunoglobulin molecules may be derived from isotype switching using well-known techniques. Immunoglobulin molecules for use in the invention may also be conjugated to other molecules for delivery. For example an immunoglobulin or fragment of the invention may form part of an antibody-drug conjugate in which the antibody is linked, via a linker, to another molecule, which may be a cytotoxin, a targeting molecule, a molecule that facilitates entry into cells, or any other molecule that has an adjunct therapeutic effect. Antibody-drug conjugates are well-known in the art. Ideally, an antibody-drug conjugate comprises an antibody or fragment identified by the screening process outlined above for its ability to selectively bind to a tumor, a linker that is stable in circulation and which may releasably bind the antibody or fragment to another molecule. The other molecule may be a cytotoxin that, upon binding of the antibody or fragment to a tumor cell, is released causing cell death. The antibody may be engineered into an IgG1, IgG2, IgG3 or IgG4, IgM or antigen binding fragments, coupled to the cytotoxin. The ADC payloads can be an anti-mitotic small molecule, DNA damaging agent, pyrrole-based kinesin spindle inhibitor, topoisomerase I inhibitors, nicotinamide phosphoribosyl inhibitor, or others known in the art. In another aspect of the invention, full-length or antigen binding fragments can be conjugated to a nucleic acid. The nucleic acid can include an antisense oligonucleotide (ASO) or a small interfering RNA (siRNA). Thus, compositions of the invention may be designed for the dual purpose of eliciting an immune response against the tumor via the cross-reactivity of the IgE-derived immunoglobulin or fragment and providing a direct cytotoxic effect to the tumor.

In addition, antibodies or fragments derived from IgE-positive B cells obtained from atopic/allergic individuals may be delivered by any appropriate means. For example, molecules may be incorporated with a nanoparticle, such as a lipid nanoparticle. Molecules as discussed herein may be conjugated to elements designed to increase bioavailability and/or serum half life. For example, a polyethylene glycol (PEG) molecule may be used to carry one or more molecules to the site of a tumor.

Antibodies or fragments for use in the invention may be produce by viral vector introduction or Cas-mediated introduction of genetic material into an appropriate host cell for in vivo production.

Tumor antigen targets for molecules of the invention include cell-surface proteins, carbohydrates, lipids, nucleic acids or any other non-protein substance on or associated with the tumor.

The invention also relates to methods for treatment, diagnosis or prognosis of cancer. Methods include administering a therapeutically effective amount of a pharmaceutical formulation that comprises engineered antibodies derived from IgE-producing B cells, or nucleic acids encoding an antibody or fragment derived from an IgE-producing B cell, specific to one or more non-tumor antigens and cross-reactive to tumor antigens. The therapeutic may be delivered via any appropriate formulation, including by vector comprising an appropriate nucleic acid sequence.

In one aspect, the invention provides methods for delivering nucleic acids encoding antibodies of the invention. Such constructs are delivered via appropriate vectors. Methods of making and delivering plasmids and vectors are well known in the art, for example Naso, M., et al., Adeno-Associated Virus (AAV) as a Vector for Gene Therapy Adeno-Associated Virus (AAV) as a Vector for Gene Therapy BioDrugs. 2017; 31(4): 317-334; and Rmamoorth, M., et al., Non-Viral Vectors in Gene Therapy—An Overview, J Clin Diagn Res. 2015 January; 9(1): GE01-GE06, each incorporated by reference herein in their entirety. Methods of the invention further include transducing, via the vector, the nucleic acids to one or more host cells and producing, via one or more transduced host cells, the cross-reactive antibody or antigen-binding portion thereof.

It should be noted that the nucleic acids, including a nucleic acid sequence encoding the antibodies of the invention and/or antigen-binding portions thereof, are derived from sequences identified from B cells, which may be isolated B cells, from an atopic or allergic individual. Methods of deriving nucleic acids (for the subsequent production of the allergen-specific antibodies) are described, for example, in International PCT Application No. PCT/US2019/032951 (published as WO 2019/222679), the disclosure of which is incorporated by reference herein in its entirety. In particular, such methods include combining single cell RNA sequencing (scRNA-seq) with functional antibody assays to elucidate mechanisms underlying the regulation of IgE and to discover high affinity, non-tumor-specific antibodies that are cross-reactive to tumor cells.

As previously described, methods of the present invention provide for the administration of a therapeutically effective amount of a pharmaceutical formulation to a subject for preventing or treating an allergic response in said subject. The formulation generally includes a composition comprising the vector and other components, such as, for example, one or more pharmaceutically acceptable carriers, adjuvants, and/or vehicles appropriate for the particular route of administration for which the composition is to be employed. The carrier, adjuvant, and/or vehicle may be suitable for injection (via a needle of the like) for intravenous, intramuscular, intraperitoneal, transdermal, or subcutaneous administration, as well as a consumable, or spray for related oral and inhalant administrations.

In another embodiment, compositions of the invention are delivered using a Cas endonuclease-mediated delivery system. The Cas endonuclease cassette using appropriate guide RNAs directed is delivered to a site of interest in cells for expression of the therapeutic antibody or fragment via insertion of a coding sequence in the host cell genome. Accordingly, administration of the pharmaceutical formulation subsequently results in in vivo production of the appropriate IgE-producing B cell-derived antibodies via viral vector introduction or Cas-mediated introduction of related genetic material into host cells. As previously noted, the antibody may include an antibody that specifically binds to any known allergen with cross-reactivity for cancer. For example, the specific allergen may include, but is not limited to, a food allergen, a plant allergen, a fungal allergen, an animal allergen, a dust mite allergen, a drug allergen, a cosmetic allergen, or a latex allergen. In some embodiments, the antibody is an antibody that specifically binds to a food allergen, such as a milk allergen, an egg allergen, a nut allergen, a fish allergen, a shellfish allergen, a soy allergen, a legume allergen, a seed allergen, or a wheat allergen. In some embodiments, the antibody specifically binds to a peanut allergen.

In aspects of the invention, the antigen-binding function of an antibody or fragment of the invention may be performed by fragments of the full-length antibody comprising the binding portion of the antibody. Examples of binding portions of antibodies include a monovalent fragment consisting of the variable light chain, variable heavy chain, and CL and CH1 constant regions (Fab fragment). Examples also include bivalent fragments comprising two Fab fragments linked by a disulfide bridge at the hinge region (F(ab′)2 fragment), fragments consisting of the variable heavy chain and CH1 constant domain (Fd fragment), fragments consisting of the variable light chain and variable heavy chain domains of a single arm of an antibody (Fv fragment), fragments consisting of a variable heavy chain domain (dAb fragment), and isolated complementarity determining region.

For fragments comprising both the variable light chain and variable heavy chain regions, the regions may be joined, using recombinant methods, by a synthetic linker that enables them to be made as a single protein chain in which the variable light chain and variable heavy chain regions pair to form monovalent molecules known as single chain Fv (scFv).

In another embodiment, an antibody or antigen binding fragment is conjugated to a label for diagnostic and therapeutic applications. Accordingly, conjugated antibodies or fragments are delivered and bind to antigenic sites on tumors for imaging via detection of the label. Optimal labels include radiolabels (as, for example, a radionucleotide), optical labels, and chemical labels.

In another embodiment, an antibody or antigen binding fragment of the invention is conjugated to drug-loaded nanoparticles to target the drug-loaded nanoparticles to tumors. Drugs or therapeutic agents incorporated into nanoparticles include small molecule toxins, cytotoxic inhibitors, nucleic acids and radionucleotides.

Cancers

The present invention is useful to treat a cancer by providing antibodies or antigen-binding portions thereof with specificity for at least one non-tumor antigen and a tumor antigen. The bispecificity of antibodies or fragments of the invention is obtained due to the fact that the antibodies are derived from IgE-producing B cells obtained from allergic/atopic individuals.

The cancer may comprise a liquid tumor or a solid tumor. Exemplary liquid tumors include leukemias and lymphomas. Further cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CIVIL), and hairy cell leukemia. Exemplary solid tumors include sarcomas and carcinomas. Cancers can arise in virtually an organ in the body, including blood, bone marrow, lung, breast, colon, bone, central nervous system, pancreas, prostate and ovary. Further cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

Treating cancer with the engineered antibodies of the present disclosure can result in a reduction in the size of a tumor, reduction in tumor volume, reduction in tumor growth rate, reduction in tumor regrowth, a decrease in number of tumors, and/or a decrease in number of metastatic lesions in other tissues or organs distant from the primary tumor site. Treating cancers with the antibodies of the present disclosure can also result in an increase in average survival time of a population of treated subjects in comparison to a population of untreated subjects or a population receiving monotherapy with a drug and not the antibodies of the present invention.

### Incorporation by Reference

References and citations to other documents, such as patents, patent applications, patent publications, journals, books, papers, web contents, have been made throughout this disclosure.

All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An antibody, or antigen-binding portion thereof, derived from an IgE-producing B cell obtained from an allergic/atopic individual with specificity for at least one tumor antigen.

2. The antibody or antigen-binding portion of claim 1, wherein said antibody is cross-reactive with at least one non-tumor antigen.

3. The antibody of claim 1, wherein said antibody is a monoclonal antibody.

4. The antibody of claim 3, wherein the monoclonal antibody includes a heavy chain variable region sequence, and a light chain variable region sequence derived from an IgE-producing human B cell associated with the at least one non-tumor antigen.

5. The antibody or antigen-binding portion thereof of claim 2, wherein the at least one nontumor antigen comprises at least one of a food allergen, a plant allergen, a fungal allergen, an animal allergen, a drug allergen, a cosmetic allergen, and a latex allergen.

6. The antibody or antigen-binding portion thereof of claim 5, wherein the one or more allergens is a food allergen selected from the group consisting of peanut, a milk allergen, an egg allergen, a tree nut allergen, a fish allergen, a shellfish allergen, a soy allergen, a legume allergen, a seed allergen, and a wheat allergen.

7. A vector comprising nucleic acid derived from an IgE-producing B cell from an atopic/allergic individual and encoding an antibody or antigen-binding portion thereof with specificity for at least one a tumor antigen.

8. The vector of claim 7, wherein the vector is a viral vector.

9. The vector of claim 7, wherein the viral vector is an adeno-associated virus (AAV).

10. A method of preventing or treating cancer, the method comprising:
administering a therapeutically effective amount of a pharmaceutical formulation comprising a vector comprising nucleic acid encoding an antibody or antigen-binding portion thereof derived from an IgE-producing B cell from an atopic/allergic individual and with specificity for at least one tumor antigen.

11. The method of claim 10, further comprising:
transducing one or more cells with nucleic acid sequence encoding a cross-reactive antibody or antigen-binding portion thereof with specificity for at least one non-tumor antigen and a tumor antigen, thereby creating one or more transduced host cells capable of expressing the cross-reactive antibody or antigen-binding portion thereof with specificity for at least one nontumor antigen and a tumor antigen.

12. (canceled)
